(19) United States
(12) Patent Application Publication
(10) Pub. No.: US 2020/0405988 A1
(43) Pub. Date: Dec. 31, 2020

(54) ORAL CARE DEVICE FOR INTUBATION

(71) Applicant: Derrick Flint, Austin, TX (US)

(72) Inventor: Derrick Flint, Austin, TX (US)

(21) Appl. No.: 16/456,873

(22) Filed: Jun. 28, 2019

(51) Int. Cl. A61M 16/04; A61M 13/00; A61C 17/10

(52) U.S. Cl. CPC A61M 16/049; A61M 2210/0625; A61C 17/10; A61M 13/003

(57) ABSTRACT

An oral care device for use during intubation. The device can include a first guard, a second guard, a first extension, a second extension, and a central pathway. The first guard can be configured for insertion into an oral cavity of a patient. The first extension and the second extension can each extend from the first guard and can together be configured to engage a dentition of a patient. The second guard can be configured to surround and engage an exterior of an oral cavity of the patient.

### BACKGROUND

Endotracheal tubes are commonly used during intubation to provide respiratory support for a patient during various procedures, such as in operative anesthesia, intensive care, and for respiratory support during a mouth, throat, or trachea obstruction. An endotracheal tube can be inserted through a mouth of a patient and into the patient's trachea to maintain an open airway and to help prevent asphyxiation. In some cases, endotracheal tubes can remain inserted to support patient respiration for days or longer.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an anterior view of an oral care assembly on a patient, in accordance with at least one example of this disclosure.

FIG. 2 illustrates a posterior-superior view of an oral care assembly, in accordance with at least one example of this disclosure.

FIG. 3 illustrates a lateral view of an oral care assembly, in accordance with at least one example of this disclosure.

FIG. 4A illustrates an anterior view of an oral care device in a first state, in accordance with at least one example of this disclosure.

FIG. 4B illustrates an anterior view of an oral care device in a second state, in accordance with at least one example of this disclosure.

FIG. 5A illustrates an anterior view of an oral care device, in accordance with at least one example of this disclosure.

FIG. 5B illustrates a superior view of an oral care device, in accordance with at least one example of this disclosure.

FIG. 6 illustrates a superior view of an oral care device, in accordance with at least one example of this disclosure.

### DETAILED DESCRIPTION

Non-ambulatory patients often require long-term intubation, such as patients requiring sedation or patients in a coma. In these cases, an endotracheal tube can be inserted through a mouth of a patient and into the patient's trachea to maintain an open airway and to help prevent asphyxiation. In some cases, the endotracheal tube can remain inserted to support patient respiration for days, weeks, or months. While the intubation replaces respiration, other bodily processes may be inactive, such as saliva production, lingual movement, and swallowing. As a result, bacteria can accumulate within the mouth (or oral cavity) of the patient which can cause oral infections, pneumonia, and can cause teeth to rot. Also, the patient's mouth can be open for long periods of time and can become dry, leading to cracks and cuts, which can be prone to infection (in part due to the bacteria-supporting environment). Current solutions include flushing and swabbing, tasks often performed by hospital staff, and while helpful, may be insufficient to prevent the complications discussed above.

The present disclosure addresses these problems by providing an oral care device including multiple guards and fluid channels. An internal guard can protect a patient's dentition and an external guard can provide a moisture seal at the patient's mouth to help maintain a moist oral environment. The moisture seal can be releasable to receive an endotracheal tube through the oral device to allow intubation while helping and can be resealable to further maintain a moist oral environment while helping to stabilize the endotracheal tube. The oral care device can also include channels and ports for receiving an oral cleaning solution and for discharging saliva and cleaning solution, which can help reduce bacteria build-up.

This overview is intended to provide an overview of subject matter of the present patent application. It is not intended to provide an exclusive or exhaustive explanation of the invention. The detailed description is included to provide further information about the present patent application.

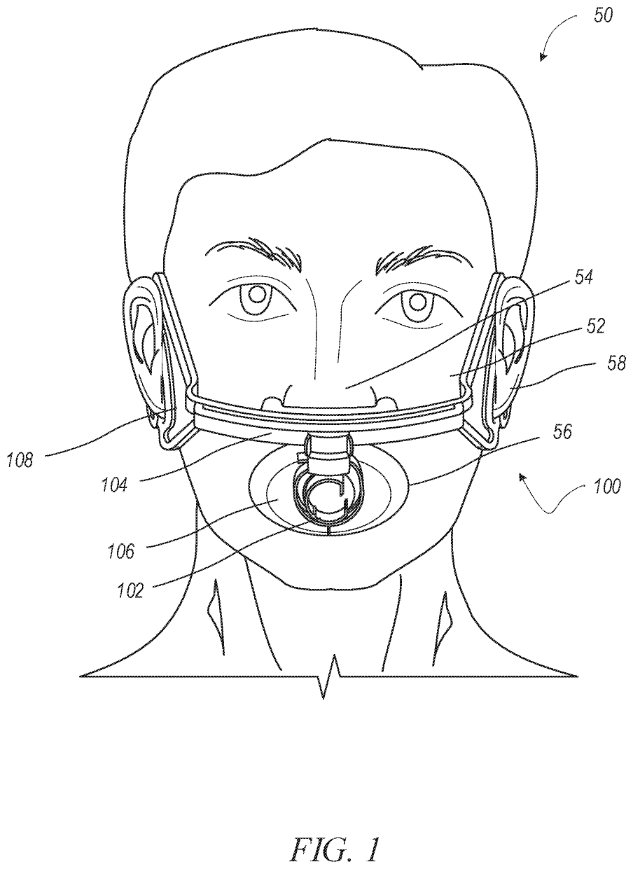

FIG. 1 illustrates an anterior view of an oral care assembly 100 on a patient 50, in accordance with at least one example of this disclosure. The oral care assembly 100 can include an endotracheal tube 102, a strap 104, and an oral care device 106.

The endotracheal tube 102 can be a tracheal tube or catheter comprised of various materials such as one or more of plastics, metals, composites, foams, or the like. The tube 102 can be insertable into a trachea of the patient 50 to establish and maintain an airway for respiration of the patient 50. The endotracheal tube 102 can be of various shapes and sizes, such as an elongate hollow cylinder. In some examples, the endotracheal tube can be flexible. Though tube 102 is discussed as being an endotracheal tube, in some examples, the tube 102 can be other types of tubes or tubes used for purposes other than tracheal insertion.

The strap 104 can be one or more pieces securable to the oral care device 106 and configured to engage one or more features of the patient 50 to secure the oral care device 106 to the patient. In some examples, the strap 104 can engage a nose 54 and face 52 of the patient and can route around ears 58 of the patient where the strap 104 can be secured behind a head of the patient 50.

The oral care device 106 can be an oral care device including one or more guards for protecting a dentition and oral cavity of the patient 50. The oral care device 106 can also include a central pathway for receiving the endotracheal tube 102 therethrough to enable intubation of the patient while the oral care device 106 is installed or in use. As discussed below in further detail, the oral care device 106 can include an internal guard to protect a dentition of the patient 50 and an external guard to provide a moisture seal at the mouth 56 of the patient 50 to help maintain a moist oral environment to help limit infection.

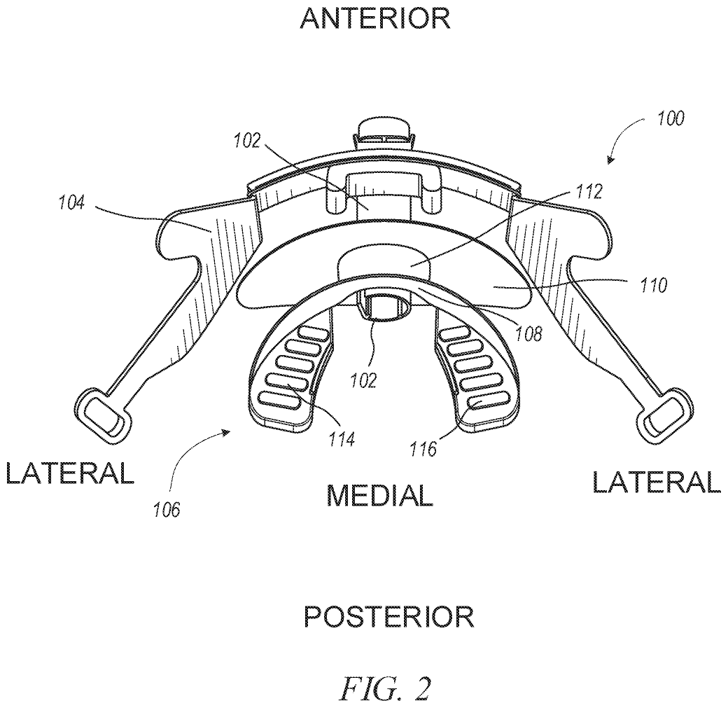

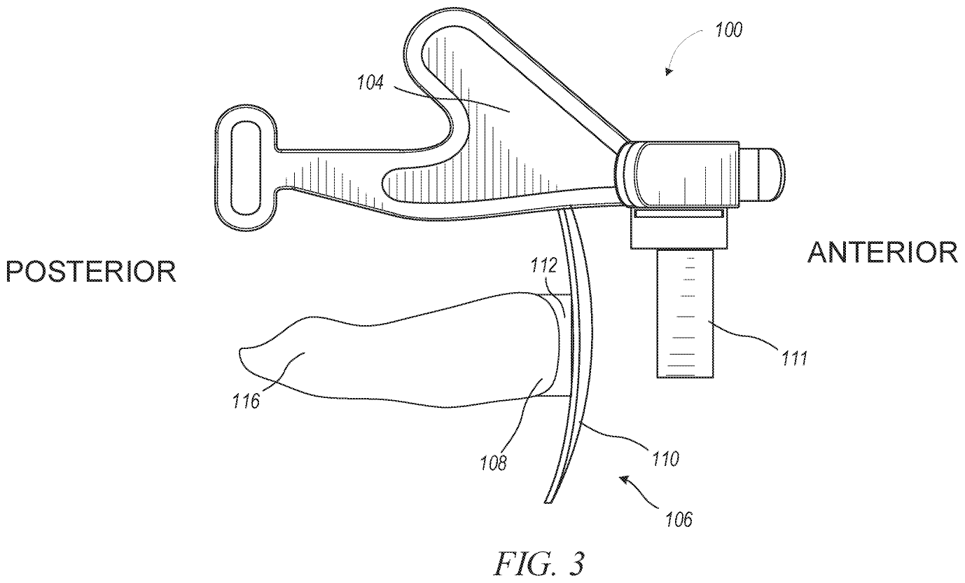

FIG. 2 illustrates a posterior-superior view of an oral care assembly 100, in accordance with at least one example of this disclosure. FIG. 3 illustrates a lateral view of an oral care assembly 100, in accordance with at least one example of this disclosure. FIGS. 2 and 3 are discussed below concurrently.

The oral care assembly 100 can include the endotracheal tube 102, the strap 104, and the oral care device 106. The oral care device 106 can include a first guard 108, a second guard 110, a central conduit 112, a first extension 114, and a second extension 116. Also shown in FIGS. 2 and 3 are orientation indicators Anterior, Posterior, Medial, and Lateral.

The first guard 108 can be a rigid or semi-rigid member made of materials such as one or more of metals, plastics, foams, elastomers, ceramics, composites, or combinations thereof. In one example, the first guard can be made of a flexible plastic material. The first guard 108 can be configured for insertion into an oral cavity of a patient (such as patient 50 of FIG. 1). The first guard 108 can be shaped to at least partially define a central pathway that can extend through the first guard 108 to receive the endotracheal tube 102 therethrough.

Similarly, the second guard 110 can be a rigid or semi-rigid member made of materials such as one or more of metals, plastics, foams, elastomers, ceramics, composites, or combinations thereof. The second guard 110 can be configured to surround and engage an exterior of the oral cavity (or mouth 56 of the patient 50, as shown in FIG. 1). The second guard 110 can be shaped to at least partially define a central pathway, which can extend through the second guard 110.

The central conduit 112 can be a rigid or semi-rigid body connecting the first guard 108 and the second guard 110 and can at least partially define the central pathway, which can extend therethrough. The central conduit 112 can include or be a part of a bite block. For example, as shown in FIG. 2, the central pathway forms a tube configured to surround the endotracheal tube 102, which can extend through the central pathway. Such a bite-block can protect the endotracheal tube 102 from being bitten, which can help limit leaks and collapsing of the endotracheal tube 102.

The first extension 114 and the second extension 116 can be a rigid or semi-rigid member comprised of materials such as one or more of metals, plastics, foams, elastomers, ceramics, composites, or combinations thereof. The first extension 114 and the second extension 116 can be connected to the first guard 108 and/or the central conduit 112 and can extend posteriorly therefrom. Each of the first extension 114 and the second extension 116 can be configured to engage a dentition of the patient (such as patient 50 of FIG. 1). The first extension 114 and the second extension 116 can engage the dentition of the patient during intubation to help limit contact between an upper and lower dentition to help reduce wear on the patient's dentition during long-term intubation.

The oral care assembly 100 can be secured to a patient before or after intubation and the oral care device 106 can be secured to the endotracheal tube either before or after intubation occurs. Further, the oral care device 106 can be inserted into the patient's oral cavity either before or after intubation occurs. In one example, the endotracheal tube 102 can be inserted into the patient's oral cavity and trachea and the oral care device 106 can be passed over the endotracheal tube 102 while the endotracheal tube 102 is inserted into the patient's trachea. Then, the first extension 114, the second extension 116, and the first guard 108 can be inserted into the patient's oral cavity with the second guard 110 abutting the patient's oral cavity.

The oral care device 106 can be inserted into the patient's oral cavity and can be secured to the endotracheal tube through a support 111 (or tube holder 111). The support 111 can be connected to the strap 104 and can be configured to support and retain the endotracheal tube in a fixed position relative to the strap 104 and the oral care device 106. The external (second) guard 110 can provide a moisture seal at the patient's mouth to help maintain a moist oral environment. As discussed in further detail below, the moisture seal can be resealable to receive the endotracheal tube 102 through the oral care device 106 to further enable installation of the oral care device 106 and endotracheal tube 102 in any order.

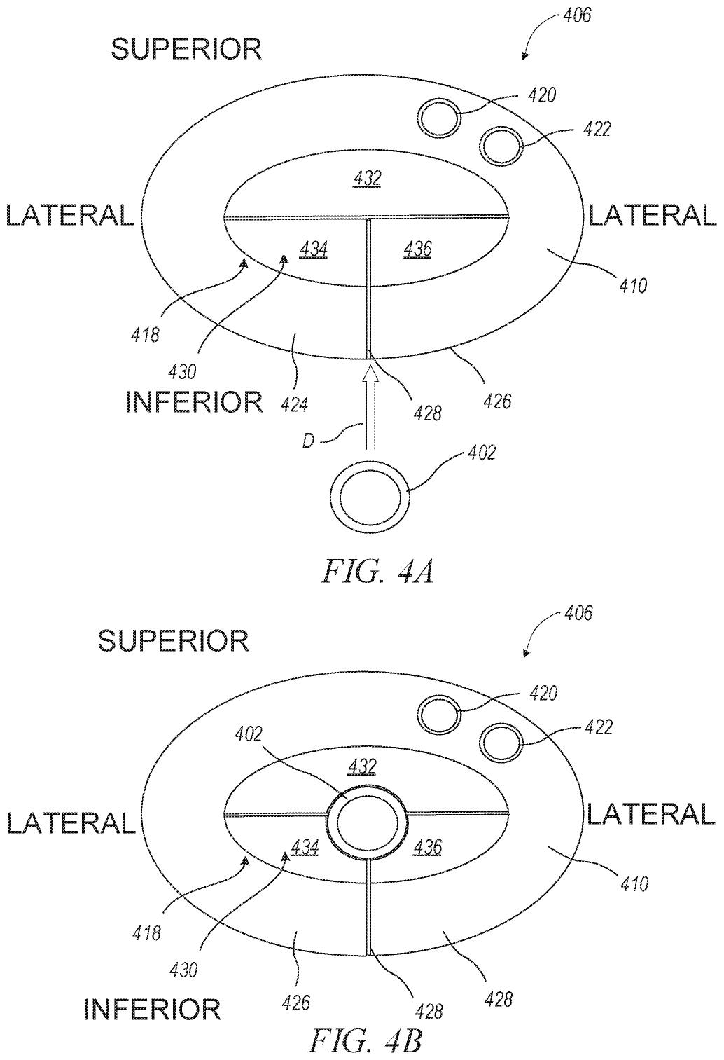

FIG. 4A illustrates an anterior view of an oral care device 406 in a first state, in accordance with at least one example of this disclosure. FIG. 4B illustrates an anterior view of an oral care device 406 in a second state, in accordance with at least one example of this disclosure. The oral care device 406 can include a moisture seal that can be releasable to receive an endotracheal tube and resealable to maintain a moist environment. Oral care devices discussed above and below can be modified to include such a moisture seal. FIGS. 4A and 4B are discussed below concurrently.

The oral care device 406 can include a second guard 410, a central pathway 418, an inlet port 420, and an outlet port 422. The second guard 410 can include a first portion 424, a second portion 426, a first seal 428, and a second seal 430. The second seal 430 can include a superior seal portion 432, an inferior seal portion 434, and an inferior seal portion 436. Also shown in FIGS. 4A and 4B are endotracheal tube 402, direction arrow D (FIG. 4A only), and orientation indicators Lateral, Superior, and Inferior.

The first portion 424 and the second portion 426 can each be portions of the second guard 410 releasably connectable at the seal 428. The seal 428 can be at an inferior portion of second guard 410, as shown in FIGS. 4A and 4B, but can be in the superior or lateral portions in other examples. The first portion 424 and the second portion 426 can form the first seal 428 by creating a butt-joint. In other examples, the first portion 424 and the second portion 426 can include interlocking features or tabs or other fastening arrangements to secure the first portion 424 to the second portion 426.

The inlet port 420 and the outlet port 422 can each be ports extending through second guard 410 and can be configured to respectively connect to a fluid supply and a fluid return, as discussed in further detail below. The inlet port 420 and the outlet port 422 can also connect to the first guard and the extensions of the oral care device where the inlet port 420 can be configured to deliver fluid to the first guard and/or the extensions and the outlet port 422 can be configured to receive fluid from the extensions and/or the first guard. Together, the inlet port 420 and the outlet port 422 can transfer fluid to and from the oral cavity of a patient to help maintain a moist environment while helping to reduce bacteria growth.

The second seal 430 can be a moisture barrier configured to form a resealable seal at an entrance to an oral cavity or mouth. The second seal 430 can be comprised of multiple seal portions connected to and extending radially inward from the second guard 410 to define an entrance of the central pathway 418. For example: the superior seal portion 432 can extend radially inward from a superior portion of the second guard 410; the inferior seal portion 434 can extend radially inward from a lateral-inferior portion of the second guard 410; and, the inferior seal portion 436 can extend radially inward from a lateral-inferior portion of the second guard 410 opposite the inferior seal portion 434. The superior seal portion 432 can meet the inferior seal portions 434 and 436 and the inferior seal portions 434 and 436 can meet each other, all to form the second seal 430, which can define the opening to the central pathway 418.

The superior seal portion 432, the inferior seal portion 434, and the inferior seal portion 436 can all be comprised of a semi-rigid or flexible material (such as plastic or rubber), allowing each of the superior seal portion 432, the inferior seal portion 434, and the inferior seal portion 436 to be able to move with respect to the guard 410 and be able to resist moisture transfer therethrough. The superior seal portion 432, the inferior seal portion 434, and the inferior seal portion 436 can also have a relatively small thickness to improve flexibility with respect to the second guard 410.

In operation of some examples, when it is desired to insert the endotracheal tube 402 into the oral care device 406, the first portion 424 and the second portion 426 can be separated at the first seal 428. Because the inferior seal portion 434 is connected to the first portion 424 and the inferior seal portion 436 is connected to the second portion 426, the inferior seal portions 434 and 436 can move together with the first portion 424 and the second portion 426, respectively, to allow separation of the inferior seal portions 434 and 436 from each other and from the superior seal portion 432 and to open the central pathway 418. Once the central pathway 418 is open, the endotracheal tube 402 can be moved in direction D to be inserted between the first portion 424 and the second portion 426 into the seal 430.

Due to their flexible properties, when the first portion 424 and the second portion 426 are released, the first portion 424 and the second portion 426 can return to meet each other to re-form the seal 428. When the endotracheal tube 402 is positioned within the seal 430, the superior seal portion 432, the inferior seal portion 434, and the inferior seal portion 436 can flex and/or fold to conform to a shape of the endotracheal tube 402 as shown in FIG. 4B, re-forming the seal 430 partially around the endotracheal tube 402 at the oral cavity. These features and functions can help maintain a moisture barrier or seal at the second guard 410 while allowing the endotracheal tube 402 to extend through the oral care device 406. The seal 430 can be easily releasable and resealable to allow repositioning of the endotracheal tube with respect to the seals 428 and 430 and removal of the oral care device from the oral cavity of the patient during intubation.

In some examples, the second seal 430 can be semi-permeable to allow for transfer of some moisture through the second seal, where the evaporation rate of the second seal 430 when wet or moist can be selected to help maintain a desired moisture environment within the oral cavity of the patient.

In some examples, the superior seal portion 432, the inferior seal portion 434, and the inferior seal portion 436 can overlap each other to help create a moisture seal at the oral cavity. In some examples, the superior seal portion 432, the inferior seal portion 434, and the inferior seal portion 436 can each contact a mouth (such as lips) of the patient to form a seal at the oral cavity of the patient.

Though the first seal 428 is shown as being on an inferior side of the guard 410, the first seal 428 can be in any position, as can the positions of the superior seal portion 432, the inferior seal portion 434, and the inferior seal portion 436. For example, the first seal 428 can be on a lateral side of the second guard aligned with a border of the superior seal portion 432 and the inferior seal portion 434.

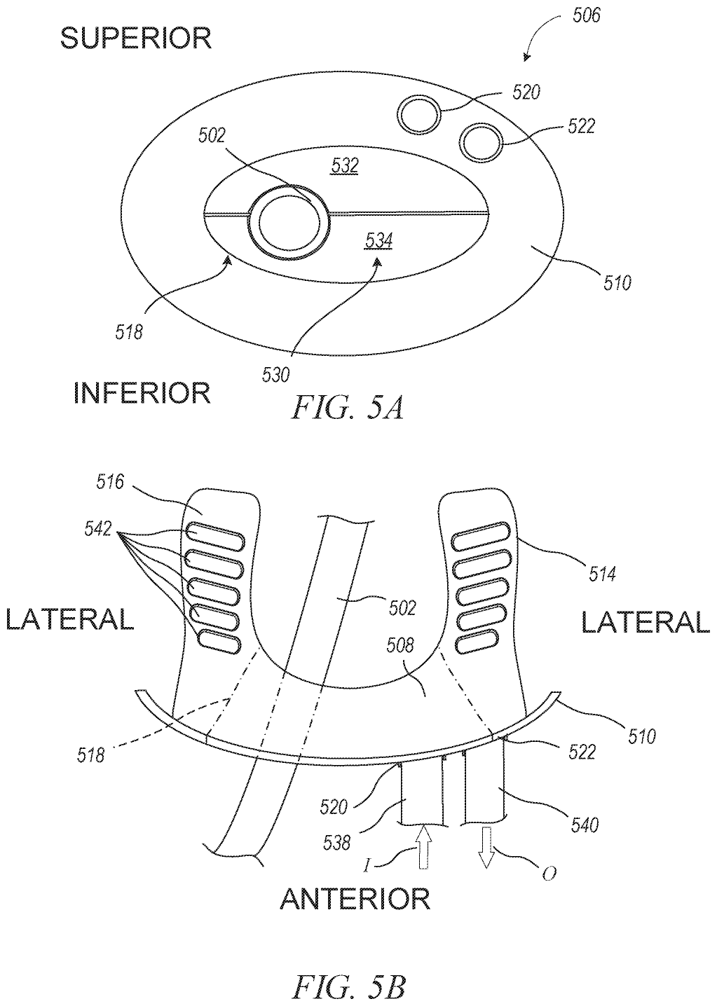

FIG. 5A illustrates an anterior view of an oral care device 506, in accordance with at least one example of this disclosure. FIG. 5B illustrates a superior view of the oral care device 506, in accordance with at least one example of this disclosure. The oral care device 506 can include a two-piece moisture seal that can be releasable to receive an endotracheal tube. Oral care devices discussed above and below can be modified to include such a moisture seal. FIGS. 5A and 5B are discussed below concurrently.

The oral care device 506 can include a first guard 508, a second guard 510, a first extension 514, a second extension 516, a central pathway 518, an inlet port 520, and an outlet port 522. Each of the first extension 514 and the second extension 516 can include bosses 542. The seal 530 can include a superior seal portion 532 and an inferior seal portion 534. Also shown in FIG. 5A are endotracheal tube 502 and orientation indicators Superior and Inferior. Also shown in FIG. 5B are endotracheal tube 502, inlet tube 538, outlet tube 540, and orientation indicators Anterior, Lateral, and Medial.

The oral care device 506 of FIGS. 5A and 5B can be similar to the oral care device 406 of FIGS. 4A and 4B discussed above, except that the seal 530 can include only two seal portions (the superior seal portion 532 and the inferior seal portion 534). The superior seal portion 532 and the inferior seal portion 534 can be flexible members coupled to and extending radially inward from the second guard 510. The superior seal portion 532 and the inferior seal portion 534 can be flexible and configured to form a seal around the endotracheal tube 502 when the endotracheal tube 502 is inserted into the central pathway.

In the example of FIG. 5A, the second guard 510 is shown without an outer seal, which can allow the second guard 510 to be relatively more rigid. In other examples, the second guard 510 can include an outer or first seal similar to the oral care device 406 in some examples, where the outer seal is aligned with the seam or abutment of the superior seal portion 532 and the inferior seal portion 534. However, the outer seal is not required as the endotracheal tube can be inserted through the seal 530 prior to intubation without separating the guard 510. Though FIG. 5A shows the guard 510 as having only two seal portions (the superior seal portion 532 and the inferior seal portion 534), the second guard 510 can have three seal portions as shown in FIGS. 4A and 4B and can have more seal portions, such as 4, 5, 6, 7, 8, 9, 10, or the like.

FIG. 5B shows how central pathway 518 can extend through the first guard 510 and the second guard 508 to extend into a patient's oral cavity for insertion into a trachea of the patient. FIG. 5B also shows bosses 542, which can extend from a surface of the extensions 514 and 516. The bosses 542 can be configured to engage the dentition of a patient to help prevent movement of the guard with respect to the dentition and the oral cavity, which can help reduce wounds such as ulcers to the oral cavity of the patient.

FIG. 5B also shows how the inlet tube 538 can be insertable into the inlet port 520 and similarly how the outlet tube 540 can be insertable into the outlet port 522. The inlet port 520 and outlet port 522 can have a press-fit engagement with the inlet tube 538 and the outlet tube 540, respectively. In other examples, the inlet port 520 and outlet port 522 can be of other engagement types, such as threaded, barbed, clamped, interlocking (fastened), slip (push), or the like.

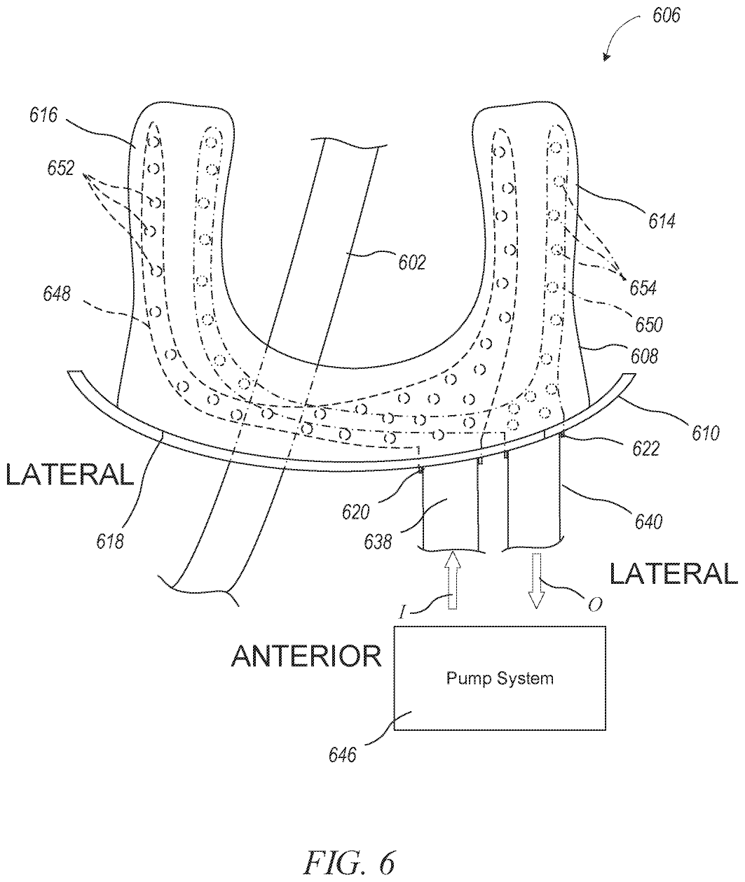

In operation, the inlet tube 538 can be configured to receive a liquid (such as a cleaning solution) in the direction indicated by arrow I for delivery to the inlet port 520. The inlet port 520 can be connected to the first guard 508 for distribution of the fluid into the oral cavity of the patient. Distribution can be performed by channels and ports of the extensions 514 and 516 (as discussed below with respect to FIG. 6) and/or can be performed through the extensions 514 and 516 themselves when the extensions 514 and 516 (and/or the guard 508) are made of a permeable (and absorbent material). In some examples, the first extension 514 and the second extension 516 can be comprised of an absorbent or super-absorbent polymer safe for use in the oral cavity of the patient. In some examples, the guard 508 and the extensions can be comprised of a sponge material or sponge-like material.

Following distribution of fluid through the guard 508 and/or the extensions 514 and 516, the guard 508 and/or the extensions 514 and 516 can absorb saliva and/or cleaning solution, which can be delivered to the outlet port 522 and ultimately to the outlet tube 540 for transfer of the liquid in a direction indicated by arrow O to a machine for disposal or processing, as discussed further below.

When the internal or first guard 508 and extensions 514 and 516 are made of an absorbent material, they can be separable from the second guard 510 allowing the extensions 514 and 516 (and the first guard 508 in some examples) to be regularly changed out by medical staff to help reduce bacteria growth in the oral cavity. In some examples, these components can be reusable after disinfection or sterilization. Also, when the internal or first guard 508 and extensions 514 and 516 are made of an absorbent material, the material can be selected to be relatively soft and conforming to the oral cavity of the patient to help limit an occurrence of ulcers.

FIG. 6 illustrates a superior view of the oral care device 606, in accordance with at least one example of this disclosure. The oral care device 606 can include supply and return channels within a guard for delivery of a fluid and collection of the fluid and saliva, which can help reduce bacteria growth of an oral cavity of a patient. Oral care devices discussed above and below can be modified to include these channels.

The oral care device 606 can include a first guard 608, a second guard 610, a first extension 614, a second extension 616, a central pathway 618, an inlet port 620, an outlet port 622, a supply channel 648, and a return channel 650. The supply channel 648 can include supply channel ports 652 and the return channel 650 can include return channel ports 654. Also shown in FIG. 6 are an endotracheal tube 602, an inlet tube 638, an outlet tube 640, a pumping system 646, and orientation indicators Anterior and Lateral.

The supply channel 648 can be a channel configured to transmit fluid extending into the guard 608, the first extension 614, and the second extension 616. The supply channel 648 can include supply channel ports 652, which can each extend from the supply channel 648 to an outer surface of any of the guard 608, the first extension 614, or the second extension 616. For example, a supply channel port 652 can extend from the supply channel 648 within the second extension 616 to a superior surface of the second extension 616 to fluidly connect the oral cavity to the supply channel 648.

Similarly, the return channel 650 can be a channel configured to transmit fluid extending into the guard 608, the first extension 614, and the second extension 616. The return channel 650 can include return channel ports 654, which can each extend from the return channel 650 to an outer surface of any of the guard 608, the first extension 614, or the second extension 616. For example, a return channel port 654 can extend from the return channel 650 within the first extension 614 to an inferior surface of the first extension 614 to fluidly connect the oral cavity to the supply channel 648.

The supply channel 648 and the return channel 650 and the ports thereof can be fluidly isolated within the guard 608, the first extension 614, and the second extension 616 to help ensure that fluid is distributed from the supply channel 648 and into the oral cavity, helping to avoid fluid bypass from the supply channel 648 to the return channel 650. In some examples, the supply channel ports 652 and return channel ports 654 can extend to opposing surfaces of the guard 608, the first extension 614, and the second extension 616 to further help reduce bypass.

The pumping system 646 can be a system commonly employed in hospital rooms configured to provide low pressure and intermittent suction from a first tube (inlet tube 638) and a positively pressurized fluid (such as a cleaning solution) out of a second tube (outlet tube 640).

In operation of some examples, the oral care device 606 can be secured within a patient's oral cavity to provide one or more moisture and/or fluid seals. The endotracheal tube 602 can be inserted through the central pathway 618 of the oral care device either before or after installation of the oral care device 606, as discussed above. The supply tube 638 can be connected to the inlet or supply port 620 and the return tube 640 can be connected to the outlet or return port 622.

The pump system 646 can then be operated to deliver a fluid, such as a cleaning solution, to the supply tube 638, which can travel through the inlet port 620 and to the supply channel 648. The supply channel 648 can distribute fluid throughout the guard 608, the first extension 614, and the second extension 616 and the fluid can be distributed to the oral cavity via the supply channel ports 652. The fluid can interact with surfaces of the oral cavity to kill bacteria and to dissolve or otherwise receive bacteria into the fluid.

During or after delivery of the fluid, the pump system 646 can supply suction to the outlet tube 640, creating a relatively negative pressure within the return channel 650 and therefore the return channel ports 654, causing fluid to flow from the oral cavity into the return channel ports 654, through the return channel 650, and to the return tube 640. Saliva within the oral cavity can follow a similar flow path. The fluid can take with it bacteria and other material from within the oral cavity, which can help reduce infection of the mouth and respiratory system (such as pneumonia).

### NOTES AND EXAMPLES

The following, non-limiting examples, detail certain aspects of the present subject matter to solve the challenges and provide the benefits discussed herein, among others.

Example 1 is an oral care device for use during intubation, the device comprising: a first guard configured for insertion into an oral cavity of a patient, the first guard shaped to at least partially define a central pathway that extends through the first guard, the central pathway configured to receive an endotracheal tube therethrough; a first extension and a second extension each extending from the first guard and together configured to engage a dentition of the patient; and a second guard configured to surround and engage an exterior of the oral cavity of the patient, the second guard shaped to at least partially define the central pathway that extends through the second guard, the second guard comprising: a first portion and a second portion couplable to form a seal at the oral cavity and configured to resealably separate to open the second guard to receive the endotracheal tube into the central pathway.

In Example 2, the subject matter of Example 1 optionally includes subject matter where the first guard includes an intraoral guard configured to engage a superior portion of the dentition.

In Example 3, the subject matter of any one or more of Examples 1-2 optionally include a central conduit connecting the first guard to the second guard, the central pathway extending through the central conduit.

In Example 4, the subject matter of any one or more of Examples 1-3 optionally include a second seal located in the second guard and connected to the first portion and the second portion of the second guard, the central pathway extending through the second seal, the second seal including a first seal portion and a second seal portion couplable to form a secondary seal at the oral cavity and configured to resealably separate to open the second seal to receive the endotracheal tube into the central pathway.

In Example 5, the subject matter of Example 4 optionally includes wherein the second seal is semi-permeable.

In Example 6, the subject matter of any one or more of Examples 4-5 optionally include wherein the second seal includes a third seal portion couplable to the first seal portion and the second seal portion to form the secondary seal at the oral cavity, and such that the first seal portion is configured to resealably separate from the second seal portion to open the secondary seal to receive the endotracheal tube into the central pathway.

In Example 7, the subject matter of any one or more of Examples 1-6 optionally include a supply channel extending into the first extension and the second extension, the supply channel configured to deliver a fluid into the oral cavity proximate the dentition.

In Example 8, the subject matter of Example 7 optionally includes a supply port connected to the supply channel and extending through the second guard, the supply port couplable to a fluid supply.

In Example 9, the subject matter of Example 8 optionally includes a return channel extending into the first extension and the second extension, the return channel fluidly separated from the supply channel, the return channel configured to receive the fluid from the oral cavity; and a return port connected to the return channel and extending through the second guard, the return port couplable to a fluid return.

In Example 10, the subject matter of any one or more of Examples 1-9 optionally include subject matter where the first portion and the second portion of the second guard are separable at an inferior portion of the second guard.

Example 11 is an oral care device configured for use with an intubation device, the device comprising: a first guard configured for insertion into an oral cavity of a patient; an extension extending from the first guard and configured to engage a dentition of the patient; a central pathway defined at least partially by the first guard, the central pathway extending through the first guard and configured to receive a tube therethrough; a second guard configured to at least partially surround and engage an exterior of the oral cavity of the patient to form a seal at the exterior of the oral cavity, the central pathway extending through the second guard, and the second guard separable to receive the tube into the central pathway; a supply channel extending into the extension, the supply channel configured to deliver a fluid into the oral cavity proximate the dentition; and a return channel extending into the extension, the return channel separated from the supply channel, and the return channel configured to receive the fluid from the oral cavity.

In Example 12, the subject matter of Example 11 optionally includes subject matter where the second guard includes a first portion and a second portion couplable to form the seal at the oral cavity and releasable to open the second guard to receive the tube into the central pathway.

In Example 13, the subject matter of any one or more of Examples 11-12 optionally include a central conduit connecting the first guard to the second guard, the central pathway extending through the central conduit.

In Example 14, the subject matter of any one or more of Examples 11-13 optionally include a supply port connected to the supply channel and extending through the second guard, the supply port couplable to a fluid supply; and a return port connected to the return channel and extending through the second guard, the return port couplable to a fluid return.

In Example 15, the subject matter of any one or more of Examples 11-14 optionally include subject matter where first guard includes a plurality of supply ports connected to the supply channel and configured to deliver the fluid from the supply channel to the oral cavity proximate the dentition.

In Example 16, the subject matter of Example 15 optionally includes subject matter where the first guard includes a plurality of return ports connected to the return channel and configured to receive the fluid from the oral cavity proximate the dentition.

Example 17 is an oral care device for use during intubation, the device comprising: a first guard configured for insertion into an oral cavity of a patient; a first extension and a second extension each extending from the first guard and together configured to engage a dentition of the patient; a central pathway extending through the first guard and configured to receive a tube therethrough; a second guard configured to surround and engage an exterior of the oral cavity of the patient, the central pathway extending through the second guard, the second guard comprising: a first portion and a second portion couplable to form a seal at the oral cavity and releasable to open the second guard to receive the tube into the central pathway; a second seal located in the second guard and connected to the first portion and the second portion of the second guard, the central pathway extending through the second seal, the second seal including a first seal portion and a second seal portion couplable to form a secondary seal at the oral cavity and configured to resealably separate to open the secondary seal to receive the tube into the central pathway; a supply channel extending into the first extension and the second extension, the supply channel configured to deliver a fluid into the oral cavity proximate the dentition; and a return channel extending into the first extension and the second extension, the return channel fluidly separated from the supply channel, the return channel configured to receive the fluid from the oral cavity.

In Example 18, the subject matter of Example 17 optionally includes a supply port connected to the supply channel and extending through the second guard, the supply port couplable to a fluid supply; and a return port connected to the return channel and extending through the second guard, the return port couplable to a fluid return.

In Example 19, the subject matter of any one or more of Examples 17-18 optionally include wherein the second guard is configured to retain fluid at the exterior of the oral cavity of the patient.

In Example 20, the subject matter of any one or more of Examples 17-19 optionally include wherein the second seal includes a third seal portion couplable to the first seal portion and the second seal portion to form the secondary seal at the oral cavity and configured to resealably separate from the first seal portion and the second seal portion to open the secondary seal to receive the tube into the central pathway.

Example 21 is an oral care device for use during intubation, the device comprising: a first guard configured for insertion into an oral cavity of a patient, the first guard shaped to at least partially define a central pathway that extends through the first guard, the central pathway configured to receive a tube therethrough; a first extension and a second extension each extending from the first guard and together configured to engage a dentition of the patient; and a second guard configured to surround and engage an exterior of the oral cavity of the patient, the second guard shaped to at least partially define the central pathway that extends through the second guard, the second guard comprising: a first portion and a second portion couplable to form a seal at the oral cavity and configured to resealably separate to open the second guard to receive the tube into the central pathway.

In Example 22, the subject matter of Example 21 optionally includes a bite block extending between the first guard and the second guard and at least partially forming the central pathway.

In Example 23, the subject matter of any one or more of Examples 21-22 optionally include wherein the first extension and the second extension are comprised of an absorbent material.

In Example 24, the subject matter of Example 23 optionally includes wherein the first extension and the second extension are comprised of a super-absorbent polymer.

In Example 25, the apparatuses or methods of any one or any combination of Examples 1-24 can optionally be configured such that all elements or options recited are available to use or select from.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An oral care device for use during intubation, the device comprising:
a first guard configured for insertion into an oral cavity of a patient, the first guard shaped to at least partially define a central pathway that extends through the first guard, the central pathway configured to receive an endotracheal tube therethrough;
a first extension and a second extension each extending from the first guard and together configured to engage a dentition of the patient; and
a second guard configured to surround and engage an exterior of the oral cavity of the patient, the second guard shaped to at least partially define the central pathway that extends through the second guard, the second guard comprising:
a first portion and a second portion couplable to form a seal at the oral cavity and configured to resealably separate to open the second guard to receive the endotracheal tube into the central pathway.

2. The oral care device of claim 1, wherein the first guard includes an intraoral guard configured to engage a superior portion of the dentition.

3. The oral care device of claim 1, further comprising:
a central conduit connecting the first guard to the second guard, the central pathway extending through the central conduit.

4. The oral care device of claim 1, further comprising:
a second seal located in the second guard and connected to the first portion and the second portion of the second guard, the central pathway extending through the second seal, the second seal including a first seal portion and a second seal portion couplable to form a secondary seal at the oral cavity and configured to resealably separate to open the second seal to receive the endotracheal tube into the central pathway.

5. The oral care device of claim 4, wherein the second seal is semi-permeable.

6. The oral care device of claim 4, wherein the second seal includes a third seal portion couplable to the first seal portion and the second seal portion to form the secondary seal at the oral cavity, and wherein the first seal portion is configured to resealably separate from the second seal portion to open the secondary seal to receive the endotracheal tube into the central pathway.

7. The oral care device of claim 1, further comprising:
a supply channel extending into the first extension and the second extension, the supply channel configured to deliver a fluid into the oral cavity proximate the dentition.

8. The oral care device of claim 7, further comprising:
a supply port connected to the supply channel and extending through the second guard, the supply port couplable to a fluid supply.

9. The oral care device of claim 8, further comprising:
a return channel extending into the first extension and the second extension, the return channel fluidly separated from the supply channel, the return channel configured to receive the fluid from the oral cavity; and
a return port connected to the return channel and extending through the second guard, the return port couplable to a fluid return.

10. The oral care device of claim 1, wherein the first portion and the second portion of the second guard are separable at an inferior portion of the second guard.

11. An oral care device configured for use with an intubation device, the device comprising:
a first guard configured for insertion into an oral cavity of a patient;
an extension extending from the first guard and configured to engage a dentition of the patient;
a central pathway defined at least partially by the first guard, the central pathway extending through the first guard and configured to receive a tube therethrough;
a second guard configured to at least partially surround and engage an exterior of the oral cavity of the patient to form a seal at the exterior of the oral cavity, the central pathway extending through the second guard, and the second guard separable to receive the tube into the central pathway;
a supply channel extending into the extension, the supply channel configured to deliver a fluid into the oral cavity proximate the dentition; and
a return channel extending into the extension, the return channel separated from the supply channel, and the return channel configured to receive the fluid from the oral cavity.

12. The oral care device of claim 11, wherein the second guard includes a first portion and a second portion couplable to form the seal at the oral cavity and releasable to open the second guard to receive the tube into the central pathway.

13. The oral care device of claim 11, further comprising:
a central conduit connecting the first guard to the second guard, the central pathway extending through the central conduit.

14. The oral care device of claim 11, further comprising:
a supply port connected to the supply channel and extending through the second guard, the supply port couplable to a fluid supply; and
a return port connected to the return channel and extending through the second guard, the return port couplable to a fluid return.

15. The oral care device of claim 11, wherein first guard includes a plurality of supply ports connected to the supply channel and configured to deliver the fluid from the supply channel to the oral cavity proximate the dentition.

16. The oral care device of claim 15, wherein the first guard includes a plurality of return ports connected to the return channel and configured to receive the fluid from the oral cavity proximate the dentition.

17. An oral care device for use during intubation, the device comprising:
a first guard configured for insertion into an oral cavity of a patient;
a first extension and a second extension each extending from the first guard and together configured to engage a dentition of the patient;
a central pathway extending through the first guard and configured to receive a tube therethrough;
a second guard configured to surround and engage an exterior of the oral cavity of the patient, the central pathway extending through the second guard, the second guard comprising:
a first portion and a second portion couplable to form a seal at the oral cavity and releasable to open the second guard to receive the tube into the central pathway;
a second seal located in the second guard and connected to the first portion and the second portion of the second guard, the central pathway extending through the second seal, the second seal including a first seal portion and a second seal portion couplable to form a secondary seal at the oral cavity and configured to resealably separate to open the secondary seal to receive the tube into the central pathway;
a supply channel extending into the first extension and the second extension, the supply channel configured to deliver a fluid into the oral cavity proximate the dentition; and
a return channel extending into the first extension and the second extension, the return channel fluidly separated from the supply channel, the return channel configured to receive the fluid from the oral cavity.

18. The oral care device of claim 17, further comprising:
a supply port connected to the supply channel and extending through the second guard, the supply port couplable to a fluid supply; and
a return port connected to the return channel and extending through the second guard, the return port couplable to a fluid return.

19. The oral care device of claim 17, wherein the second guard is configured to retain fluid at the exterior of the oral cavity of the patient.

20. The oral care device of claim 17, wherein the second seal includes a third seal portion couplable to the first seal portion and the second seal portion to form the secondary seal at the oral cavity and configured to resealably separate from the first seal portion and the second seal portion to open the secondary seal to receive the tube into the central pathway.
